(19) United States
(12) Patent Application Publication
(10) Pub. No.: US 2024/0158844 A1
(43) Pub. Date: May 16, 2024

(54) DUAL-PROBE METHOD FOR FLUORESCENCE QUANTITATIVE PCR

(71) Applicant: Fuzhou Maixin Biotech Co., Ltd. China, Fuzhou (CN)

(72) Inventors: Yiwei HUANG, Fuzhou (CN); Chun MENG, Fuzhou (CN); Jing HONG, Fuzhou (CN); Wenxiao MA, Fuzhou (CN); Xiaoya WANG, Fuzhou (JP); Qixin LIN, Fuzhou (JP)

(21) Appl. No.: 18/179,192

(22) Filed: Mar. 6, 2023

(30) Foreign Application Priority Data
Nov. 16, 2022 (CN) 202211461083.5

(51) Int. Cl. C12Q 1/6858; C12Q 1/6818

(52) U.S. Cl. CPC C12Q 1/6858; C12Q 1/6818; C12Q 1/6851; C12Q 2600/156

(57) ABSTRACT

A dual-probe method for fluorescence quantitative PCR (Polymerase Chain Reaction) is disclosed. The method involves the use of a Taqman dual-probe detection system with the same pair of primers. The dual probes includes a detection probe and a reference probe. The detection probe targets the wild-type sequence at the hotspot mutation site, which can cover multiple adjacent mutations. The reference probe targets the wild-type sequence adjacent to the target sequence. Both probes share the same pair of upstream and downstream primers. This method uses a single reaction qPCR (quantitative PCR) method, which can simultaneously detect multiple adjacent mutations. It effectively saves tissue samples and greatly shortens the testing period. Moreover, the close proximity of the target and reference sequences enhances the reliability and accuracy of the results, making it widely applicable in various fields.

### CROSS REFERENCE OF RELATED APPLICATIONS

This application claims priority of Chinse Patent Application No. 202211461083.5, filed on Nov. 16, 2022, entitled “DUAL-PROBE METHOD FOR FLUORESCENCE QUANTITATIVE PCR,” in the China National Intellectual Property Administration (CNIPA), the entire contents of which are hereby incorporated by reference in their entireties.

### FIELD OF THE DISCLOSURE

The present disclosure relates to the technical field of molecular biology technology, especially to a dual-probe method for fluorescence quantitative PCR.

### BACKGROUND OF THE DISCLOSURE

Polymerase Chain Reaction (PCR) is a revolutionary method developed by Kary Mullis in the 1980s in the field of molecular biology. Its principle is to use the ability of DNA polymerase to synthesize a new DNA strand complementary to the provided template strand. Since DNA polymerase can only add nucleotides to pre-existing 3′-OH groups, primers need to be added to initiate the reaction in the amplification system. At the end of the PCR reaction, specific sequences are enriched, and billions of copies (amplicons) can be obtained.

The development of PCR technology has advanced to the third generation digital PCR technology. However, the second generation fluorescence quantitative PCR technology, with its advantages such as easy operation and simple result interpretation, still remains the mainstream application technology in the market. The generation of fluorescent signals is crucial in this technology. Real-time fluorescence PCR technology based on TaqMan fluorescent probes is the most widely used in clinical diagnosis in China. The thermostable DNA polymerase Taq not only has 5′—>3′ polymerase activity, but also has 5′—>3′ exonuclease activity for nucleotide sequences that bind to the target sequence encountered during polymerase extension. During amplification, when the primer extends by the polymerase reaction of Taq DNA polymerase to a probe that has already bound to the target nucleic acid sequence, the 5′—>3′ exonuclease activity of Taq DNA polymerase will degrade the probe into small fragments. The TaqMan fluorescent probe is labeled with a fluorescent reporting group at its 5′ end and a fluorescent quencher group at its 3′ end. According to the principle of fluorescence resonance energy transfer, the emitted fluorescence of the fluorescent reporting group is quenched because it is in close proximity to the quencher group in the intact probe. When the probe is degraded, the fluorescent reporter group and the quencher are separated, and the fluorescence is emitted. During PCR, Taq DNA polymerase uses its 5′ exonuclease activity to cleave the oligonucleotide probe that binds to the target sequence, eventually releasing the fluorescence.

qPCR typically requires two pairs of primers and probes to amplify the target gene and reference gene, respectively. The reference gene is usually a conservative segment of a housekeeping gene located far from the target gene, and its expression level varies greatly between different tissues and cell cycles. After PCR, the difference in fluorescence signals between the target gene and the reference gene is used for interpretation. However, this interpretation result is easily affected by changes in the copy number of the reference gene. Test reagents that rely on qPCR methods usually require a large number of primer or probe screenings for each mutation during the R&D (research and development) stage, which consumes samples and has a long testing cycle, thereby increasing the testing cost.

### SUMMARY OF THE DISCLOSURE

In view of the aforementioned problems, the present disclosure provides a dual-probe method for fluorescence quantitative PCR, which can be applied to the detection of various gene mutations.

In order to achieve the object, following solution is provided.

A dual-probe method for fluorescence quantitative PCR (Polymerase Chain Reaction) is provided. The dual-probe includes a detection probe and a reference probe. Each of the probes is labeled with a different fluorescent marker.

The detection probe targets the wild-type sequence at a hotspot mutation site, while the reference probe targets the wild-type sequence adjacent to a target sequence. Both of the detection probe and the reference probe share the same pair of upstream and downstream primers.

In some embodiments, the dual-probe method uses a Taqman dual-probe detection system with the same pair of primers.

In some embodiments, fluorescent groups carried by the detection probe and the reference probe are one of FAM, VIC (HEX), CY3, CY5, ROX. The fluorescent groups carried by the detection probe and the reference probe are different. The quencher groups carried by the detection and the reference probe are one of BHQ1, BHQ2, TAMRA, MGB NFQ.

Compared with the prior art, the advantages of the present disclosure are as follows.

The dual-probe method for fluorescence quantitative PCR provided by this disclosure uses single-reaction qPCR, which enables simultaneous detection of multiple adjacent mutations, effectively saving tissue samples and greatly shortening the test cycle. The fact that the target sequence and reference sequence are closely located increases the credibility and accuracy of the results, and this method has broad application prospects.

### BRIEF DESCRIPTION OF DRAWINGS

In order to clarify the embodiments of the present disclosure, the accompanying drawings required for embodiments will be briefly described below. It should be understood that the following drawings only illustrate some embodiments of the present disclosure and should not be deemed as limiting the scope of patent protection. Other drawings may be obtained by skilled persons in the art without inventive effort based on the drawings of the present disclosure.

FIG. 1 shows a PCR amplification curve of the sample in Example 1 according to the present disclosure.

FIG. 2 shows a PCR amplification curve of the sample in Example 2 according to the present disclosure.

FIG. 3 shows a PCR amplification curve of the sample in Example 3 according to the present disclosure.

FIG. 4 shows a PCR amplification curve of the sample in Example 4 according to the present disclosure.

FIG. 5 is a schematic diagram of the detection principle according to the present disclosure.

### DETAILED DESCRIPTION OF THE DISCLOSURE

In order to clarify the objects, technical solutions and advantages of the embodiments of the present disclosure, the technical solutions in the embodiments of the present invention will be described clearly and completely below. It is evident that the described embodiments are a part of the embodiments of the present disclosure, rather than all of them. Based on the embodiments of the present disclosure, all other embodiments obtained by skilled artisans in the art without creative effort are within the scope of protection of the present invention.

The present invention utilizes a Taqman dual-probe detection system with the same pair of primers. The dual probes includes a detection probe and a reference probe, each labeled with a different fluorescent marker. The detection probe targets the wild-type sequence at the hotspot mutation site, which can cover multiple adjacent mutations, while the reference probe targets the wild-type sequence adjacent to the target sequence. The two probes share the same pair of upstream and downstream primers.

Furthermore, the detection probe and the reference probe separately carries a fluorescent group, different from each other. The fluorescent group may be one of FAM, VIC (HEX), CY3, CY5, or ROX. Both the detection probe and the reference probe carry a quencher group. The quencher group carried by the detection probe and the reference probe may be one of BHQ1, BHQ2, TAMRA, or MGB NFQ.

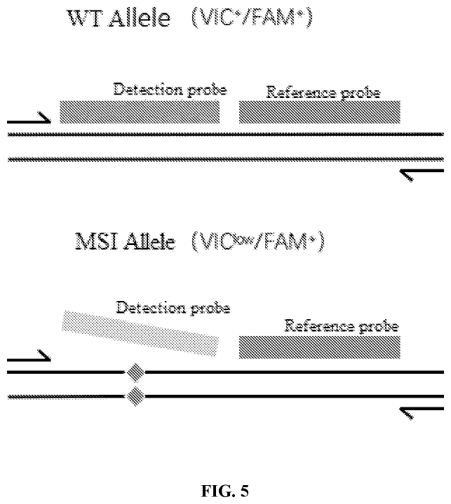

As shown in FIG. 5, when there is no mutation sequence in the template, the detection probe perfectly matches the template and releases a fluorescence signal, while the reference probe releases a normal fluorescence signal (VIC⁺/FAM⁺). When there is a mutation sequence in the template, the detection probe does not perfectly match the template, and the fluorescence signal weakens, while the reference probe releases a fluorescence signal (VIC^{low}/FAM⁺). This method can simultaneously detect multiple adjacent mutations, saving samples, and using only one pair of primers, reducing the reaction cost. The target sequence is close to the reference sequence, which improves the reliability and accuracy of the results and has broad application prospects.

Experimental Part

Main reagents: TAKARA-RR390, water;

Main instruments: handheld centrifuge, vortex oscillator, fluorescence quantitative PCR instrument;

The components of the reaction system are shown in Table 1 as below.

TABLE 1

| Component | Volume (μL) |
|---|---|
| Premix Ex Taq | 12.5 |
| Upstream primer (10 mM) | 1.25 |
| Downstream primer (10 mM) | 1.25 |
| Detection probe (10 mM) | 0.625 |
| Reference probe (10 mM) | 0.5 |
| ddH₂O | 5.875 |
| Template (1000 copies/μL) | 3 |
| Total | 25 |

### Example 1: Detection of EGFR T790M Mutation in Simulated Sample with a Mutation Frequency of 5%

The reaction system was prepared based on Table 1. In the reaction system,

the sequence of the upstream primer:
5′-GCGAAGCCACACTGACGT-3′;
the sequence of the downstream primer:
5′-AAGGGCATGAGCTGCGT-3′;
the sequence of the detection probe:
5′-(VIC)-GTGGACAACCCCCACGTGT-(MGB NFQ)-3′;
and
the sequence of the reference probe:
5′-(6-FAM)-CTGCTGGGCATCTGCCT-(MGB NFQ)-3′.

The detection template was a T790M simulated sample with a mutation frequency of 5% prepared by mixing wild-type human genomic DNA with a plasmid containing the EGFR T790M mutation gene fragment. The wild-type sample was human genomic DNA with the same copy number as the simulated sample.

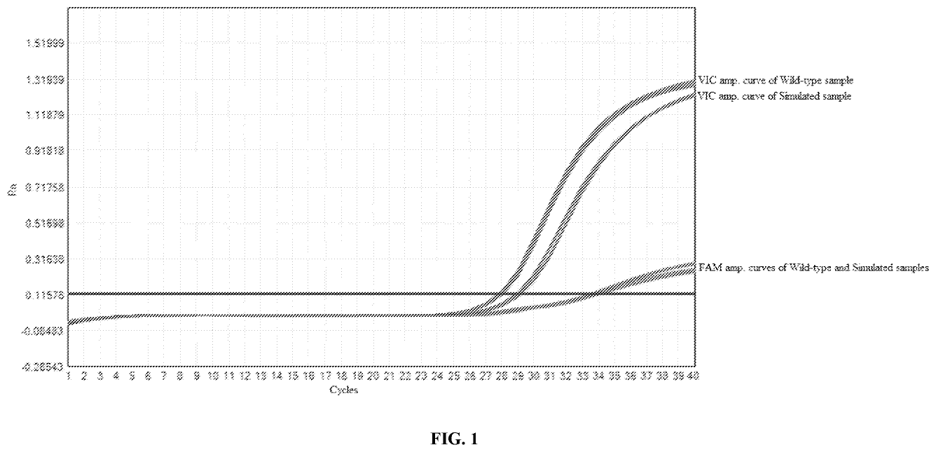

As shown in FIG. 1, there is a significant difference in the amplification curve between the simulated and control samples, indicating that the reaction system can distinguish between wild-type and mutant samples. Table 2 shows the Ct values obtained from this test. It can be seen from the table that the ΔCt between the two fluorescent channels in the wild-type sample is 4.53, while that in the simulated sample is 5.96. The difference between the ΔCt values of the two samples, namely ΔΔCt, is 1.43. The significance analysis of the ΔCt values of the two samples showed a p-value <0.0001, indicating that the reaction system can distinguish between wild-type and mutant samples.

TABLE 2

| Fluorescent channel | | FAM | VIC | ΔCt |
|---|---|---|---|---|
| Wild-type sample | Parallel 1 | 33.97 | 27.99 | 5.98 |
| | Parallel 2 | 33.55 | 27.87 | 5.68 |
| | Parallel 3 | 34.09 | 28.03 | 6.06 |
| | Parallel 4 | 33.88 | 27.78 | 6.1 |
| | Mean | — | — | 5.96 |
| Simulated sample | Parallel 1 | 33.53 | 28.89 | 4.64 |
| | Parallel 2 | 33.47 | 28.92 | 4.55 |
| | Parallel 3 | 33.54 | 29.15 | 4.39 |
| | Parallel 4 | 33.66 | 29.14 | 4.52 |
| | Mean | — | — | 4.53 |

### Example 2: Detection of KRAS G12D Mutation in a Simulated Sample with a Mutation Frequency of 5%

According to Table 1, the reaction system was prepared as following:

the sequence of the upstream primer:
5′-CTGAAAATGACTGAATATAAACTTGTGGTA-3′;
the sequence of the downstream primer:
5′-TCTATTGTTGGATCATATTCGTCCAC-3′;
the sequence of the detection probe:
5′-(VIC)-AGCTGGTGGCGTAGGC-(MGB NFQ)-3′;
and
the sequence of the reference probe:
5′-(6-FAM)-AGTGCCTTGACGATACAGCT-(MGB NFQ)-3′.

The detection template was a G12D simulated sample with a mutation frequency of 5% prepared by mixing wild-type human genomic DNA with a plasmid containing the KRAS G12D mutation gene fragment. The wild-type sample was human genomic DNA with the same copy number as the simulated sample.

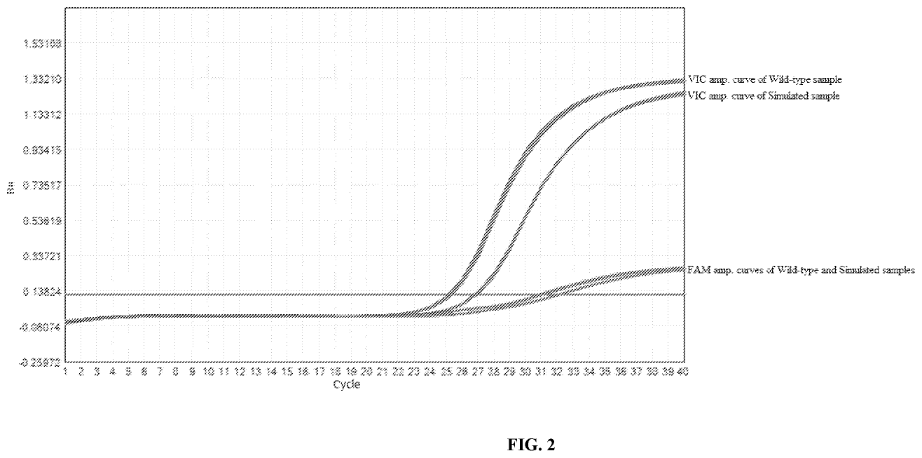

As shown in FIG. 2, there was a significant difference between the amplification curves of the simulated sample and the control sample, indicating that the reaction system can distinguish between wild-type and mutation samples. Table 3 shows the Ct values of this test. It can be seen from the table that the ΔCt between the two fluorescence channels of the wild-type sample is 5.24, and the ΔCt between the two fluorescence channels of the simulated sample is 5.75. The difference between the ΔCt values of the two samples, namely ΔΔCt, is 0.51. Significance analysis of the ΔCt values of the two samples showed a p-value <0.0001, indicating that the reaction system can distinguish between wild-type and mutation samples.

TABLE 3

| Fluorescent channel | | FAM | VIC | ΔCt |
|---|---|---|---|---|
| Wild-type sample | Parallel 1 | 30.91 | 25.2 | 5.71 |
| | Parallel 2 | 30.82 | 25.15 | 5.67 |
| | Parallel 3 | 31.13 | 25.31 | 5.82 |
| | Parallel 4 | 30.93 | 25.15 | 5.78 |
| | Mean | — | — | 5.75 |
| Simulated sample | Parallel 1 | 32.03 | 26.75 | 5.28 |
| | Parallel 2 | 32.11 | 26.89 | 5.22 |
| | Parallel 3 | 32.15 | 26.92 | 5.23 |
| | Parallel 4 | 32.15 | 26.91 | 5.24 |
| | Mean | — | — | 5.24 |

### Example 3: Detection of KRAS G12C Mutation in a Simulated Sample with a Mutation Frequency of 5%

The reaction system is prepared based on Table 1. The sequences of the upstream and downstream primers, and the detection and reference probes are the same to Example 2. The detection template was a G12C simulated sample with a mutation frequency of 5% prepared by mixing wild-type human genomic DNA with a plasmid containing the KRAS G12C mutation gene fragment. The wild-type sample was human genomic DNA with the same copy number as the simulated sample.

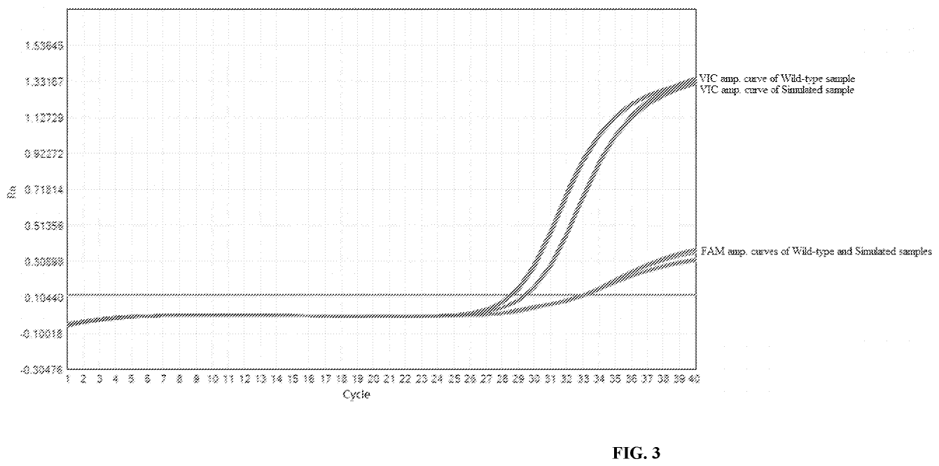

As shown in FIG. 3, there was a significant difference between the amplification curves of the simulated sample and the control sample, indicating that the reaction system can distinguish between wild-type and mutation samples. Table 4 shows the Ct values of this test. It can be seen from the table that the ΔCt between the two fluorescence channels of the wild-type sample is 4.66, and the ΔCt between the two fluorescence channels of the simulated sample is 3.62. The difference between the ΔCt values of the two samples, namely ΔΔCt, is 1.04. Significance analysis of the ΔCt values of the two samples showed a p-value <0.0001, indicating that the reaction system can distinguish between wild-type and mutation samples.

TABLE 4

| Fluorescent channel | | FAM | VIC | ΔCt |
|---|---|---|---|---|
| Wild-type sample | Parallel 1 | 33.20 | 28.60 | 4.6 |
| | Parallel 2 | 33.24 | 28.64 | 4.6 |
| | Parallel 3 | 33.11 | 28.52 | 4.59 |
| | Parallel 4 | 33.27 | 28.44 | 4.83 |
| | Mean | — | — | 4.66 |
| Simulated sample | Parallel 1 | 33.08 | 29.50 | 3.58 |
| | Parallel 2 | 33.10 | 29.45 | 3.65 |
| | Parallel 3 | 32.97 | 29.38 | 3.59 |
| | Parallel 4 | 33.01 | 29.36 | 3.65 |
| | Mean | — | — | 3.62 |

### Example 4: Detection of BRAF V600E Mutation in a Simulated Sample with a Mutation Frequency of 5%

According to Table 1, the reaction system was prepared as following:

the sequence of the upstream primer:
5′-TCATAATGCTTGCTCTGATAGGA-3′;
the sequence of the downstream primer:
5′-CTGTTCAAACTGATGGGACCC-3′;
the sequence of the detection probe:
5′-(VIC)-CTACAGAGAAATCTCGA TGGA-3′;
and
the sequence of the reference probe:
5′-(6-FAM)-TCTTCATGAAGACCTCACAGT-3′.

The detection template was a V600E simulated sample with a mutation frequency of 5% prepared by mixing wild-type human genomic DNA with a plasmid containing the BRAF V600E mutation gene fragment. The wild-type sample was human genomic DNA with the same copy number as the simulated sample.

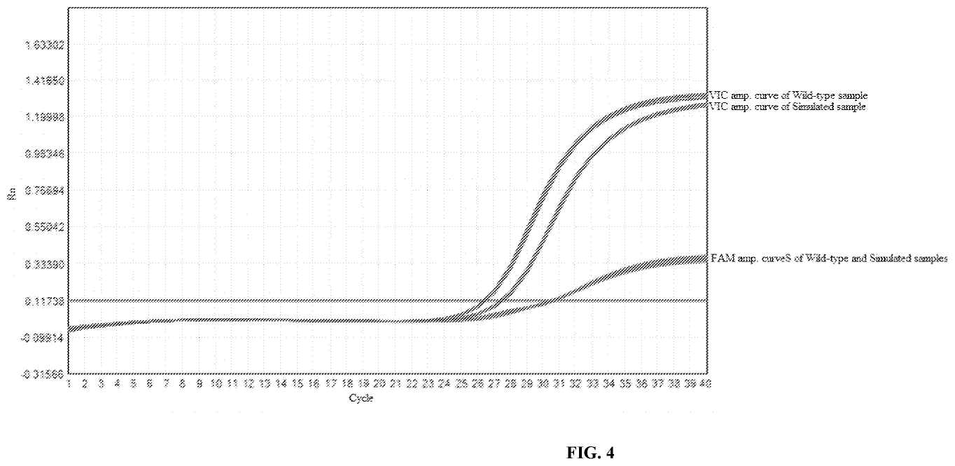

As shown in FIG. 4, there was a significant difference between the amplification curves of the simulated sample and the control sample, indicating that the reaction system can distinguish between wild-type and mutation samples. Table 5 shows the Ct values of this test. It can be seen from the table that the ΔCt between the two fluorescence channels of the wild-type sample is 3.06, and the ΔCt between the two fluorescence channels of the simulated sample is 4.24. The difference between the ΔCt values of the two samples, namely ΔΔCt, is 1.18. Significance analysis of the ΔCt values of the two samples showed a p-value <0.0001, indicating that the reaction system can distinguish between wild-type and mutation samples.

TABLE 5

| Fluorescent channel | | FAM | VIC | ΔCt |
|---|---|---|---|---|
| Wild-type sample | Parallel 1 | 30.67 | 26.47 | 4.2 |
| | Parallel 2 | 30.55 | 26.34 | 4.21 |
| | Parallel 3 | 30.65 | 26.44 | 4.21 |
| | Parallel 4 | 30.68 | 26.36 | 4.32 |
| | Mean | — | — | 4.24 |
| Simulated sample | Parallel 1 | 30.57 | 27.45 | 3.12 |
| | Parallel 2 | 30.53 | 27.51 | 3.02 |
| | Parallel 3 | 30.43 | 27.37 | 3.06 |
| | Parallel 4 | 30.46 | 27.42 | 3.04 |
| | Mean | — | — | 3.06 |

The above description is merely embodiments of the present disclosure and does not limit the scope of the patent. Any equivalent structural or procedural modifications made using this disclosure and its accompanying drawings, or direct or indirect application to other relevant arts, are also included within the scope of the present disclosure.

What is claimed is:

1. A dual-probe method for fluorescence quantitative PCR (Polymerase Chain Reaction), wherein the dual-probe comprises a detection probe and a reference probe; each of probes which is labeled with a different fluorescent marker;
the detection probe targets the wild-type sequence at a hotspot mutation site, while the reference probe targets the wild-type sequence adjacent to a target sequence; both of the detection probe and the reference probe share the same pair of upstream and downstream primers.

2. The dual-probe method according to claim 1, wherein the dual-probe method uses a Taqman dual-probe detection system with the same pair of primers.

3. The dual-probe method according to claim 1, wherein the detection probe and the reference probe each carries a fluorescent group selected from the group consisting of FAM, VIC (HEX), CY3, CY5, and ROX; the fluorescent group carried by the detection probe and the reference probe are different; the detection and the reference probe each carries a quencher group selected from the group consisting of BHQ1, BHQ2, TAMRA, and MGB NFQ.
